(19) United States
(12) Patent Application Publication
(10) Pub. No.: US 2005/0158251 A1
(43) Pub. Date: Jul. 21, 2005

(54) Agents for bleaching teeth

(76) Inventors: Carlo Bolis, Chur (CH); Alexander Karl Huwig, Horgen (CH); Reto Grimm, Sargans (CH); Volker M. Rheinberger, Vaduz (LI)

Correspondence Address: Joseph M. Noto;Nixon Peabody LLP, Clinton Square, P.O. Box 31051, Rochester, NY, 14603-1051, US

(21) Appl. No.: 10/935,813

(22) Filed: Sep. 8, 2004

(30) Foreign Application Priority Data
Jan. 15, 2004 (DE) 20 2004 000 552.2

(51) Int. Cl. A61K 7/16; A61K 7/20

(52) U.S. Cl. 424/49; 424/53

(57) ABSTRACT

Agents in varnish form for bleaching teeth based on 70 to 90 wt.-% solvent, 2 to 20 wt.-% pharmaceutically acceptable oxidant and 8 to 12 wt.-% alkyl cellulose.

The invention relates to compositions for brightening teeth which contain a pharmaceutically acceptable oxidant as active component.

Many foods and semi-luxury products, such as tea, coffee, red wine and tobacco, can lead to discolorations of the teeth. Certain drugs also, such as for example tetracycline or the intake of large amounts of fluoride can cause discoloration of the teeth. Because the mouth, as an eye-catching feature in the centre of the face, determines outward appearance to a large degree, such discolorations are often felt to spoil the image and ways and means of dealing with this are increasingly of interest.

A method which is often used involves the bleaching of the teeth with concentrated hydrogen peroxide solution (30 to 37%). To promote the oxidative action of the peroxide, heat and light are used. Although this method succeeds relatively quickly, it requires a large outlay and can be carried out only by trained personnel.

U.S. Pat. No. 4,032,627 discloses pigment-containing varnishes for masking discolorations. These varnishes have a low mechanical load-bearing capacity and must be reapplied practically every day.

U.S. Pat. No. 5,290,566 discloses agents in gel form for the brightening of teeth which contain 22 to 32 wt.-% of carbamide peroxide as active component. These gels are applied to a strip, which is then fitted onto the teeth to be treated. This strip must be worn for 6 hours every day, until the desired degree of bleaching has been achieved.

Compositions in paste form are known from U.S. Pat. No. 5,171,564 which contain abrasives as well as carbamide peroxide.

U.S. Pat. No. 5,425,953 discloses liquid polymer compositions which, as well as an oxidant such as carbamide peroxide, contain a water-soluble cellulose polymer and preferably also a stabilizer such as calcium disodium edetate. The compositions are applied to the teeth once to three times daily and form there, after the evaporation of the solvent, a film which, releases the oxidant and which decomposes within 1 hour.

Finally bleaching agents in the form of varnishes based on carbamide peroxide are known from U.S. Pat. No. 6,083,421, which are intended to adhere to the surface of the teeth for more than 1 hour. Preferred film formers are polyvinyl butyral, coumaron resin and schellac.

Bleaching agents in varnish form are in general easy to handle, and their application causes the patients relatively little trouble. Disadvantages of known bleaching varnishes are however that their bleaching action is often only small, they display low adhesiveness and they are sensitive to moisture, so that there is a need for further improved, film-forming bleaching agents for teeth.

According to the invention this problem is solved by oral compositions which contain
(i) 70 to 90 wt.-% solvent,
(ii) 2 to 20 wt.-% pharmaceutically acceptable oxidant and
(iii)8 to 12 wt.-% alkyl cellulose.

According to one version the compositions additionally contain, as well as the named components, a pharmaceutically acceptable acid, preferably phosphoric acid, (H₃PO₄), pyrophosphoric acid (H₄P₂O₇) and in particular citric acid. The acid is preferably used in a quantity of 0.05 to 5.0 wt.-%, in particular 0.5 to 5 wt.-%, especially preferably 0.3 to 0.6 wt.-% .All percentages, unless otherwise specified, relate to the overall mass of the composition.

It was found that small amounts of acid condition the surface of the tooth, i. e. start to dissolve plaque, hydroxylapatite and toothpaste residues and make it easier for the varnish to infiltrate such residues. Thanks to the acid a strong and uniform adhesion of the varnish to the surface of the tooth is thus ensured, but without damaging the tooth. At the same time a uniform wetting of the surface of the tooth is achieved, which along with a good adhesion ensures a uniform brightening of the teeth. The consequence of a non-uniform wetting of the surface or a partial detachment of a poorly adhering varnish is that the peroxide compound acts for varying lengths of time on different areas of the surface of the teeth, which can give the teeth an undesirably stained appearance.

As a further optional component the compositions according to the invention preferably also contain dexpanthenol, preferably in a quantity from 0.1 to 1.0 wt.-%, particularly preferably 0.2 to 0.5 wt.-%. Dexpanthenol is a provitamin which counteracts irritations and inflammations of the mucous membrane of the mouth which can be triggered by the peroxide compound, the solvent or the acid. Moreover dexpanthenol promotes the healing of already existing wounds. Compositions according to the invention which contain dexpanthenol are thus also suitable for the brightening of teeth where there is gingivitis and are better compatible than conventional agents.

Moreover further components, such as aromatics, flavouring and/or sweeteners can be added to the composition. These components are each preferably used in a quantity of 0.01 to 0.5 wt.-%, particularly preferably 0.02 to 0.2 wt.-% and quite particularly preferably 0.03 to 0.06 wt.-%.

Preferred aromatics and flavourings are methyl salicylate, sassafras wood oil, clove oil, sage oil, eucalyptus oil, marjoram oil, menthol and preferably mint oil, i.e. peppermint oil and/or spearmint oil. Particularly preferred are synthetic mint oils and mint aromas, in particular the oils and aromas marketed by Haarmann & Reimer under the name OPTAMINT®. Other suitable aromas are strawberry, raspberry, banana, cherry, caramel, pear, apple, lemon and peach aromas as well as orange oil and grapefruit aroma.

Saccharin sodium, sodium cyclamate, xylitol, aspartame and similar are particularly suitable as sweeteners. Saccharin sodium is particularly preferred.

Preferred pharmaceutically acceptable oxidants are sodium perborate, chlorine dioxide and pharmaceutically acceptable peroxide compounds, such as for example organic peroxides, preferably methyl peroxide, ethyl peroxide and in particular glycerol peroxide or benzyl peroxide. Particularly preferred peroxides are hydrogen peroxide and in particular carbamide peroxide. Carbamide peroxide is preferably used in a quantity from 4 to 20 wt.-%, particularly preferably 5 to 10 wt.-% and quite particularly preferably in a quantity from 5.3 to 6.5 wt.-%. Hydrogen peroxide is preferably used in a quantity from 2 to 16 wt.-%, particularly preferably 5.5 to 13 wt.-% and most preferably 5.5 to 12 wt.-%, measured as H₂O₂.

Hydrogen peroxide is preferably used in the form of a commercially available hydrogen peroxide solution in water with an H₂O₂ concentration of 34.5-36.5%. For higher hydrogen peroxide concentrations an aqueous H₂O₂-solution with a concentration of 48.8-50.6%, also commercially available, is preferably used.

The most preferred oxidant is carbamide peroxide, which is hydrolized to urea and hydrogen peroxide in the presence of water. The formed urea has the advantage that its action is pH-neutralizing. The released hydrogen peroxide diffuses into the tooth enamel and the dentin, where it breaks down into water and oxygen upon oxidation. Through the diffusion of the hydrogen peroxide into the tooth enamel the chemical equilibrium between carbamide peroxide and its decomposition products is destroyed, which results in the further release of hydrogen peroxide from the carbamide peroxide. With the varnish according to the invention it is guaranteed that the hydrogen peroxide diffuses practically completely into the tooth enamel and is not, as in conventional varnishes, lost in large amounts in saliva and soft tissue.

An important aspect of the invention is the use of alkyl cellulose as varnish former. Preferably a water-insoluble alkyl cellulose is used, i.e. alkyl cellulose with a solubility of at most 0.1 g alkyl cellulose per 100 g water at room temperature. Particularly preferred is ethyl cellulose, in particular ethyl cellulose with an average degree of substitution (DS) of >1.5, particularly preferably >2.0, in particular 2.0 to 2.6, and quite particularly preferably 2.3 to 2.4. In cellulose every glucose unit has three free hydroxyl groups. If all three hydroxyl groups of all the glucose units of the cellulose are substituted by alkoxyl groups, the degree of substitution (DS) is 3. If only some of the hydroxyl groups of the glucose units are substituted by alkoxyl groups, the degree of substitution is correspondingly lower. The degree of substitution DS indicates the average number of substituted hydroxyl groups per bound glucose unit. Quite particularly preferred is ethyl cellulose which has an ethoxyl content of 45 to 50%. The alkyl cellulose is preferably used in a quantity of 9 to 10 wt.-%.

The alkyl cellulose more preferably has a viscosity of 50 to 150 mPas, particularly preferably 80 to 110 mPas, quite particularly preferably 90 to 100 mPas, measured at 25°C. with an Ubbelohde viscometer on a 5 wt.-% solution in toluene/ethanol 80:20 (v/v).

The use of alkyl cellulose and in particular of ethyl cellulose as varnish former gives the compositions according to the invention a certain water tolerance, i.e. the action of the composition is not adversely affected by contact with saliva for example. Should the varnish come into contact with saliva after application to the tooth, which most often cannot be ruled out when used by the patient himself, the varnish is not rinsed off, but there is a precipitation of the varnish former, as a result of which a protective layer is developed which protects the varnish layer from penetration by the saliva and protects against detachment from the tooth. At the same time a washing-out of the oxidant by the saliva is prevented and it is guaranteed that the oxidant diffuses into the tooth enamel after the application of the composition. This is a significant advantage in particular when using water-soluble peroxide compounds. The compositions according to the invention thus guarantee a noticeable brightening of the teeth at relatively low concentrations of oxidant and shorter treatment times. By contrast, with conventional bleaching agents a fast washing-out of water-soluble peroxide compounds is often observed, so that a bleaching action is achieved that is only small in relation to the oxidant concentration. A low concentration of the frequently aggressive bleaching agents is a great advantage in the case of agents which are intended for application by the patient himself with regard to security of application and possible side-effects.

Preferred solvents are diethyl ether, acetone, preferably ethyl acetate and in particular ethanol. Mixtures of one or more of these solvents can be used. Moreover the solvent can contain up to 30 wt.-% of water, relative to the total quantity of solvent, i.e. to the quantity of water and other solvent. When using carbamide peroxide the water content is preferably under 12 wt.-%, particularly preferably under 2 wt.-%, anhydrous compositions being most preferred.

The compositions according to the invention are colourless-to-white varnishes, which according to a preferred version are neutral, i.e. they have a pH value in the range from 5.5 to 8, preferably 6.5 to 7.5, measured on an aqueous dispersion of the composition (1 to 25 wt.-%). The varnishes do not drip, do not pull threads on application and can be precisely applied with a brush. They dry within about 1 minute.

Acidic compositions preferably have a pH value of 1 to 6, particularly preferably 2 to 4 and quite particularly preferably 2 to 3.

The compositions can according to one version also contain dyes which have no negative influence on the bleaching action, for example carmin and carthamin. By using dyed compositions it is easier to ensure that the surface of the teeth is completely wetted. The compositions according to the invention preferably contain no abrasives.

Compositions preferred according to the invention contain the following components:
(i) 70.0 to 90.0 wt.-%, in particular, 80 to 88 wt.-%, quite particularly 84 to 86 wt.-% solvent,
(ii) 2 to 16 wt.-% oxidant, in particular 5.3 to 6.5 wt.-% carbamide peroxide or 5.5 to 13 wt.-% hydrogen peroxide,
(iii)8.0 to 12.0 wt.-%, in particular 9 to 10 wt.-% alkyl cellulose, preferably ethyl cellulose,
(iv) 0 to 5.0 wt.-%, in particular 0.5 to 5 wt.-%, particularly preferably 0.3 to 0.6 wt.-% acid, preferably citric acid,
(v) 0 to 1.0%, in particular 0.2 to 0.5% dexpanthenol,
(vi) 0 to 0,5 wt.-%, in particular 0 to 0.2 wt.-%, particularly preferably 0.01 to 0.05 wt.-% flavouring and/or aromatic, in particular mint oil or mint aroma.

A particularly preferred bleaching agent based on hydrogen peroxide has the following composition:
(i) 55 to 72 wt.-% ethanol,
(ii) 5.5 to 13.0 wt.-% hydrogen peroxide,
(iii) 8.5 to 9.5 wt.-% ethyl cellulose,
(iv) 15 to 28 wt.-% water,
(v) 0.2 to 0.5 wt.-% dexpanthenol and
(vi) 0.01 to 0.06 wt.-% aroma.

The water content here is relative to the overall mass of the composition, but account is also to be taken of the above-defined maximum water content relative to the total quantity of solvent.

A quite particularly preferred bleaching agent based on carbamide peroxide has the following composition:
(i) 79 to 85 wt.-% ethanol,
(ii) 5.3 to 6.5 wt.-% carbamide peroxide,
(iii) 8.5 to 9.5 wt.-% ethyl cellulose,
(iv) 0 or 0.3 to 0.6 wt.-% citric acid,
(v) 0.2 to 0.5 wt.-% dexpanthenol and
(vi) 0.01 to 0.06 wt.-% aroma.

The compositions according to the invention are prepared by mixing the components with each other in the desired quantities, until a homogenous, spreadable solution is obtained.

For application the teeth to be treated are first thoroughly cleaned and dried, for example by blowing with an air jet or by dabbing with a tissue. Then the composition according to the invention is applied to the surface of the teeth with an applicator, for example with a small brush, and left to dry. The drying can be accelerated by blowing with an air jet. Upon the evaporation of the solvent a colourless-to-white or optionally dyed film forms, which adheres firmly to the surface of the tooth and preferably stays in the mouth for a period of 15 to 30 minutes. During this phase the patient should speak as little as possible and not take any liquid or solid nourishment. When the action period has ended the varnish can be peeled off like a film or removed with a wet toothbrush (without toothpaste). The total application time and frequency is based on the desired bleaching result. A twice daily application over a period of fourteen days is recommended. Should the desired result already be achieved before the end of this period, the treatment can be discontinued or else extended.

Saliva contains the enzyme catalase which catalyzes the disproportionation of hydrogen peroxide accompanied by formation of water and oxygen. Catalase can thus effect a decomposition of the hydrogen peroxide contained in the bleaching agents, without this being able to penetrate the tooth enamel and bleach dyes there. Catalase can also be problematic if saliva for example is brought into the bleaching agent receptacle by the brush and causes a decomposition of the oxidant there. It was found, that compositions according to the invention based on carbamide peroxide show no noteworthy change in the carbamide peroxide concentration even after the addition of 8 vol.-% saliva over a period of 1 week, which indicates remarkable insensitivity to saliva of the compositions according to the invention.

For reasons of hygiene the compositions according to the invention are however preferably made available in packaging in which the applicator does not come into contact with the actual contents. This is also advantageous with regard to a possible precipitation of the varnish former when there is contact with saliva. A preferred packaging unit contains a receptacle, preferably a tube, with the composition according to the invention, a receptacle, preferably a small dish, for receiving the quantity of bleaching agent required for a single treatment of the teeth and an applicator such as a brush for example. To apply the composition, the necessary quantity of bleaching agent is transferred from the tube into the dish and then applied to the teeth with the applicator. In this way a contact of the composition in the tube with the applicator that is used is avoided.

A particular problem when determining the effectiveness of bleaching agents for teeth is that generally expensive tests are required on extracted human teeth or teeth of bovines. According to the invention a test system was developed, which allows an assessment of the bleaching effect in a simple way. Discolorations of the teeth are often attributable to aromatic compounds which, upon bleaching, are converted into colourless, saturated hydrocarbons. However it is known that certain aromatic compounds, such as for example the dye o-dianisidine, form coloured products, upon oxidation by hydrogen peroxide. O-dianisidine forms a stable, dark-brown-coloured cation which can be visually detected and thus allows the oxidative effect of peroxides to be demonstrated. The test according to the invention uses this reaction to assess the effect of bleaching agents.

To carry out the test, an aromatic compound which forms coloured products upon the oxidation by hydrogen peroxide, is introduced into a gellable solution and left to gel. The bleaching agent to be examined is applied in a certain quantity to a testpiece, preferably made from porcelain, glass, ceramic, glass-ceramic, and particularly preferably from a dental glass-ceramic and left to dry. Cylindrical testpieces are particularly suitable, for example with a diameter of approximately 1 cm. These coated testpieces are then applied to the gel, the coated side facing the gel. Peroxide contained in the bleaching agent diffuses into the gel and reacts there with the aromatic compound, accompanied by the development of a circular, dyed aureole around the testpieces. The diameter of the aureole is proportional to the quantity of peroxide which is released from the bleaching agent. The test allows an assessment of the speed and the quantity of the release of hydrogen peroxide. It is suitable for examining compositions which contain hydrogen peroxide, carbamide peroxide, methyl peroxide or ethyl peroxide as oxidizing component.

Particularly suitable as aromatic compound are compounds from the group of the phenols, aminophenols, aromatic diamines, indophenols and leucodyes. o-Dianisidine is quite particularly preferred. o-Dianisidine has a brownish coloration, which upon oxidation by peroxide changes to a dark brown. Because the reaction of peroxide with o-dianisidine proceeds only relatively slowly, the enzyme peroxidase, which catalyzes this reaction, is preferably also added to the agar. Another preferred aromatic compound is 2,2′-azino-bis(3-ethyl-benzthiazolin-6-sulphonic acid (ABTS).

A particularly preferred gelling solution has the following composition:
(a) 1 to 30 wt.-%, preferably 5 to 25 wt.-% and particularly preferably 10 to 20 wt.-% gelling agent;
(b) 0.01 to 0.1 wt.-%, preferably 0.015 to 0.05 wt.-% and particularly preferably 0.02 to 0.03 wt.-% aromatic compound, preferably o-dianisidine;
(c) optionally 0.001 to 0.01 wt.-%, preferably 0.0015 to 0.003 wt.-% peroxidase;
(d) buffer solution, preferably NaK phosphate buffer (ad 100 wt.-%)

The agar solution is preferably poured into petri dishes and left to gel.

Preferred gelling agents are cellulose derivatives, starch derivatives, polysaccharides, gelatins and silica gel, in particular agarose, anionic, acid heteropolysaccharides, such as gellan, and in particular agar-agar.

In the following the invention is explained in more detail with reference to embodiments in conjunction with the figures, where:

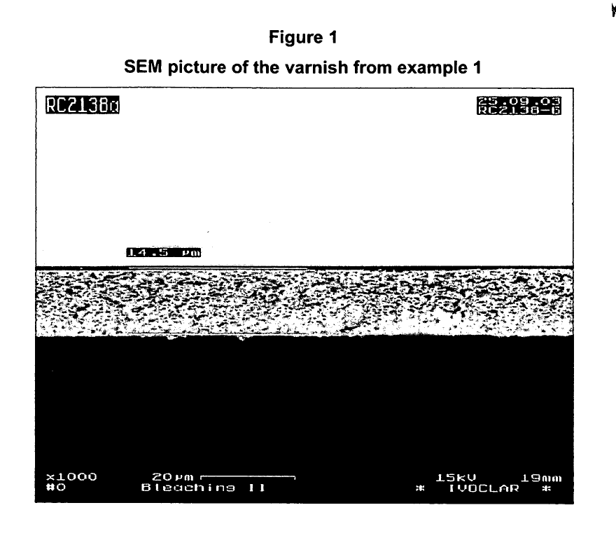

FIG. 1 shows a scanning electron microscopic picture of a varnish according to the invention after drying, which is applied to a glass microscope slide.

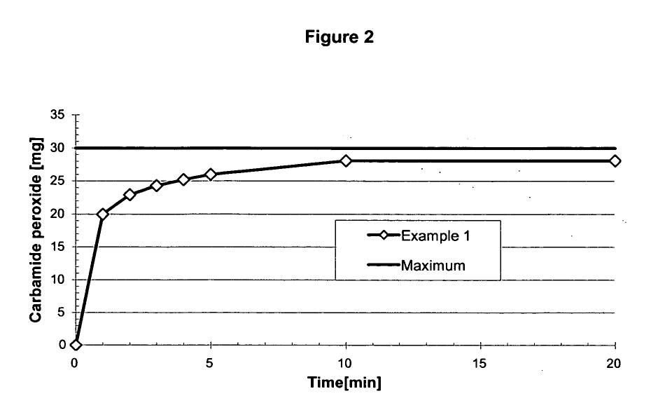

FIG. 2 shows a graphic representation of the temporal course of the release of carbamide peroxide from a varnish according to the invention.

### EMBODIMENTS

### EXAMPLE 1

### Preparation of a Bleaching Agent Based on Carbamide Peroxide

108 ml anhydrous ethanol were placed in a beaker and added to 9 g ethyl cellulose. The mixture was stirred, until there was a homogeneous solution. To this mixture 5.9 g pure carbamide peroxide, 0.3 g dexpanthenol and 0.1 g mint aroma (OPTAMINT®, Haarmann & Reimer) were added successfully and stirred up uniformly. A colourless-to-white varnish was obtained.

Chemical and physical data of the varnish:

| | |
|---|---|
| Appearance: | viscous, opaque, colourless |
| | liquid |
| Odour: | Peppermint |
| Viscosity (at 100 s⁻¹): | 2.50-4.00 Pa * s |
| Density: | 0.83-0.84 g/ml |
| pH value | 6.5-7.5 |
| (1-25% dispersed in water): | |
| Flash point: | 13° C. |

### EXAMPLE 2

### Storage Stability of the Varnish from Example 1

In order to determine the storage stability of the varnish from example 1, it was kept in a 100-ml glass bottle at room temperature for a period of 52 weeks. At the start and at the end of the examination period the oxidant content (carbamide peroxide content) was determined titrimetrically. After 52 weeks a carbamide peroxide content of 5.12% was measured, the titrimetrically determined initial concentration was 6.23%. Measurements were taken according to U. R. Kunz (1990): Grundlagen der Quantitativen Analyse [Fundamentals of Quantitative Analysis], Thieme-Verlag, Stuttgart, 3rd Edition. The varnish thus proved to be sufficiently storage-stable.

### EXAMPLE 3

### Ascertaining the Layer Thickness of the Varnish After Drying

The varnish from example 1 was applied with a brush uniformly in one layer to a glass microscope slide and left to dry. After drying the varnish layer was examined under a scanning electron microscope. A layer thickness of 10 to 20 μm was measured (FIG. 1). At 1000-times magnification it can be seen that the varnish forms a very uniform, closed layer with clearly structured texture.

### EXAMPLE 4

### Preparation of an Agar Solution for Measuring the Bleaching Action

In order to be able to simulate the process of the teeth bleaching, an enzyme-based agar test was established, with which the release of the hydrogen peroxide can be tracked.

The agar was prepared as follows:
1. Stirring of 50 mg o-dianisidine and 3 g agar-agar into 190 ml 50 mM NaK-phosphate buffer, pH7; for optimized distribution of the o-dianisidine the mixing took place using an ultrasound bath.
2. 10 minutes' autoclaving at 100° C. of the bottle which is lightly shut by a cap.
3. After the cooling to 50° C., addition of 3 mg peroxidase accompanied by stirring, previously dissolved in 10 ml buffer and added through a sterile filter using a Luer-Lock syringe.
4. Pouring of the agar into petri dishes (diameters 9 cm and 15 cm). Care is to be taken to fill the Petri dishes evenly. The filling quantity was 15 g in petri dishes of 9 cm diameter, 40 g in petri dishes of 15 cm diameter.

The agar plates can be stored upside down at 8° C. for up to two weeks.

Exactly 15 mg of the bleaching agent to be tested (bleaching varnish from example 1) were applied to a testpiece made of dental ceramic (d=1 cm, Tetric Flow, Ivoclar Vivadent AG), left to dry and the testpiece laid with the coated side on the surface of the agar. A release of carbamide peroxide occurred with subsequent hydrolysis to hydrogen peroxide and urea in the agar which mainly consisted of water. The released hydrogen peroxide effects a brown coloration of the agar, which was measured out using a calliper gauge until the size of the circle was retrograde.

The brown coloration is due to the fact that, in the agar, electrons are transmitted from the chromogen o-dianisidine onto hydrogen peroxide, which is reduced to water. The oxidized chromogen forms a stable colour varnish, the formation of which is proportional to the quantity of hydrogen peroxide applied and which can be very well recognized by its intensely brown colour. Hydrogen peroxide oxidizes o-dianisidine only very slowly, however, so that the enzyme peroxidase was additionally added to the agar as a biocatalyst. The agar is used to solidify the liquid test medium, in order to be able to track the release in a targeted way.

The test allows bleaching agents to be examined in series within hours.

### EXAMPLE 5

### Quantitative Determination of the Peroxide Release

In order to measure the release of the carbamide peroxide from the varnish, a testpiece coated with the composition from example 1 was placed in a petri dish and covered with a layer of water. After various intervals samples were taken and the concentration of the corresponding peroxide was determined with an enzymatic test. In this test peroxidase is used as catalyst and 2,2′-azino-bis(3-ethyl-benzthiazolin-6-sulphonic acid) (ABTS) as chromogen (Werner W., Rey H. G., Wielinger H. (1970): Uber die Eigenschaften eines neuen Chromogens fur die Blutzuckerbes-timmung nach der GOD/POD-Methode, [The Properties of a new chromogen for blood-sugar determination by the GOD/POD, Method]Z. Anal. Chem. 252, 224-228; Michal G., Möllering H., Siedel J. (1983): Chemical design of indicator reactions for the visible range, in: Bergmeyer H. U. (editor), Methods of enzymatic analysis, Vol. 1, Verlag Chemie, Weinheim, 197-232). In the photometric test hydrogen peroxide oxidizes the chromogen ABTS in a succeeding indicator reaction catalyzed by peroxidase to an intensely blue-green dye, the concentration of which was determined by direct photometric measurement and the concentration of which was proportional to the hydrogen peroxide concentration. Measurement took place at 420 nm, the extinction coefficient of ABTS⁺ at 420 nm is 43.2 ml μmol^{—1} cm⁻¹.

ABTS is a heterocyclic azine which with two higher oxidation steps constitutes a reversible redox system bound by two single-electron transfers. In the first oxidation step the colour-less-to-slightly light-green azine is oxidized to a blue-green radial cation (ABTS⁺) . The second oxidation step from radial cation to dark-purple azodication occurs only when there is a large excess of the oxidant and in a strongly acid environment.

In detail the measurement was carried out as follows:

A standard curve was plotted, solutions with four different concentrations of hydrogen peroxide being prepared: 0.010 mg/ml, 0.013 mg/ml, 0.020 mg/ml and 0.025 mg/ml. These concentrations led to extinction values in the range 0.1-0.7, the linear range, in which the increase in extinction is directly proportional to the reduction of HP to water.

960 μl ABTS solution (2.08 mM ABTS in 50 mM potassium phosphate buffer, pH 6.0) and 20 μl peroxidase (400 U/ml in 50 mM potassium phosphate buffer, pH 6.0) were placed in a cuvette, placed in the specimen holder of the photometer and measured for 10 s. After this the peroxide solution to be measured was added, stirred briefly to mix thoroughly and measured again. The extinction was tracked over 2 minutes.

The measured quantities of released peroxide are plotted graphically cumulatively against time (FIG. 2). It was found, that more than 80% of the peroxide was released within the first 5 minutes, 90% within the first 10 minutes. After 15 minutes the carbamide peroxide was practically completely dissolved out from the varnish.

This method is very well suited in particular to quick measurement of the hydrogen peroxide concentration as well as for release studies.

### EXAMPLE 6

### Measurement of the Catalase Sensitivity

In order to measure the sensitivity to catalase of compositions according to the invention, 2.5 vol.-% and 8 vol.-% saliva were added to the varnish from example 1 respectively and incubated for 10 days at room temperature. The saliva contained a physiologically significant quantity of catalase. It was found that the carbamide peroxide concentration remained constant through the addition of 2.5 vol.-% saliva within the period of the study. In the specimen mixed with 8 vol.-% saliva the carbamide peroxide concentration remained unchanged for approximately 7 days.

In order to check, whether a catalase activity in the saliva and a decomposition of carbamide peroxide can be recorded with. the method used, further tests were carried out in which saliva was additionally added to commercially available catalase from Aspergillus niger. Here a clear decomposition of carbamide peroxide was recorded.

### EXAMPLE 7

### Demonstration of the Bleaching Action of the Varnish from Example 1

To measure the bleaching effect of the varnish from example 1 an internal clinical in vivo study was carried out with: 5 volunteer subjects.

A general medical history was taken from all subjects and dental results compiled. A professional teeth cleaning was carried out on all subjects before the start of the treatment. Only after the teeth cleaning was the initial situation documented and the current tooth colour determined using the chromascope colour scale and photographically documented (frontal overview picture with chromascope reference specimen in the picture).

Before every varnish application, soft deposits were removed from all teeth surfaces with small nylon brushes and prophylaxis paste. The teeth to be treated (15-25 and 35-34) were then drain-dried with an air blower and dental pump. To make the teeth to be treated more easily accessible a lip-cheek holder was used (according to Dr. Horvath/Spandax from Hager & Werken).

The varnish was applied by brush, uniformly in one layer to the surface of the teeth. The varnish had a very good consistency and was able to be applied precisely with the brush, did not drip and did not pull threads. It formed a uniform layer and also wetted the approximal areas very well. When the application had ended the lip-cheek holder was left for a further 60 seconds in the mouth of the patient, in order to ensure sufficient drying of the material. Only at the end of this period was the lip-cheek holder removed. The patient was asked not to touch the teeth with the tongue for the duration of the action time. After 15 minutes' action time the material was removed with a small nylon brush and water spray and the mucous membrane was examined for irritations. This procedure was repeated once daily until the desired bleaching result had set in, but not for longer than over a period of 4 weeks, or 20 treatments.

During every fifth session, the current situation was documented. For this the current tooth colour was determined according to the chromascope colour scale and photographically documented. After the last application the current situation was again documented. The current tooth colour was determined and photographically documented. In further follow-up checks, it was evaluated whether the bleaching result remains stable.

The bleaching effect was recorded at the earliest after a period of approximately 5 days (5 applications). In the great majority of the patients a definitive brightening of the teeth set in after approximately 10 applications.

The brightening of the teeth was very uniform, with a slight emphasis on the incisal half. The subjects were treated with the bleaching agent on average approximately 14 times (average: 13.8±3.9 applications). Although the initial colour of the subjects was on average very bright, there was an average increase of clearly more than one brightness step on the chromascope scale (average: 1.6±0.5 brightness steps). In one case after 15 applications there was a two-step brightening (from 130 to 110). The brightening remained over the test period of 6 months.

The patients found the varnish to be neutral in terms of odour and taste and felt almost no bothersome effects from the varnish.

### EXAMPLE 8

### Preparation of a Bleaching Agent Based on Hydrogen Peroxide

89 ml anhydrous ethanol were placed in a beaker and mixed with 9.0 g ethyl cellulose. The mixture was stirred until there was a homogeneous solution. This was added successively to 20.0 g hydrogen peroxide solution (30%), 0.3 g dexpanthenol and 0.1 g mint aroma (Optamint® spearmint aroma 218921, Haarmann & Reimer) and uniformly stirred. An opaque varnish was obtained.

Chemical and physical data of the varnish:

| | |
|---|---|
| Appearance: | viscous, opaque, colourless |
| | liquid |
| Odour: | Peppermint |
| Viscosity (at 100 s⁻¹): | 1.5-2.5 Pa * s |
| Density | 0.86-0.88 g/ml |
| pH value | 5.5-6.5 |
| (1-25% dispersed in water): | |

1. Oral composition containing:
(i) 70-90 wt.-% solvent,
(ii) 2-20 wt.-% pharmaceutically acceptable oxidant and
(iii) 8-12 wt.-% alkyl cellulose, in each case relative to the overall mass of the composition.

2. Composition according to claim 1, which additionally contains (iv) pharmaceutically acceptable acid.

3. Composition according to claim 2, which additionally contains citric acid.

4. Composition according to claim 2, which contains 0.05-5.0 wt.-% acid, relative to the overall mass of the composition.

5. Composition according to claim 1, which additionally contains (v) dexpanthenol.

6. Composition according to claim 5, which contains 0.1 to 1.0 wt.-% dexpanthenol, relative to the overall mass of the composition.

7. Composition according to claim 6, which additionally contains (vi) at least one of an aromatic, flavouring and sweetener.

8. Composition according to claim 1, which contains at least one of ethanol, diethyl ether, ethyl acetate and acetone as solvent.

9. Composition according to claim 8, which contains ethanol as solvent.

10. Composition according to claim 1, which contains as alkyl cellulose an alkyl cellulose with a water solubility of at most 0.1 g in 100 g water at room temperature.

11. Composition according to claim 1, which contains alkyl cellulose with a viscosity of 50 to 150 mPas, measured at 25° C. with an Ubbelohde viscometer in a 5 wt.-% solution in toluene/ethanol 80:20 (v/v).

12. Composition according to claim 10, which contains ethyl cellulose as an alkyl cellulose.

13. Composition according to claim 7, which contains mint oil as an aromatic.

14. Composition according to claim 1, which contains a peroxide compound as a pharmaceutically acceptable oxidant.

15. Composition according to claim 7, which contains
(i) 70.0 to 90.0 wt.-% ethanol,
(ii) 2 to 16.0 wt.-% oxidant,
(iii) 8.0 to 12.0 wt.-% ethyl cellulose,
(iv) 0 to 5.0 wt.-% citric acid,
(v) 0 to 1.0 wt.-% dexpanthenol,
(vi) 0 to 0.5 wt.-% aroma, in each case relative to the overall mass of the composition.

16. Composition according to claim 14, which contains carbamide peroxide as peroxide compound.

17. Composition according to claim 16, which contains
(i) 79 to 85 wt.-% ethanol,
(ii) 5.3 to 6.5 wt.-% carbamide peroxide,
(iii) 8.5 to 9.5 wt.-% ethyl cellulose,
(iv) 0 or 0.3 to 0.6 wt.-% citric acid,
(v) 0.2 to 0.5 wt.-% dexpanthenol and
(vi) 0.01 to 0.06 wt.-% aroma, in each case relative to the overall mass of the composition.

18. Composition according to claim 14, which contains a hydrogen peroxide as peroxide compound.

19. Composition according to claim 18, which contains up to 30 wt.-% water, relative to the total quantity of solvent.

20. Composition according to claim 18, which contains
(i) 55 to 72 wt.-% ethanol,
(ii) 5.5 to 13.0 wt.-% hydrogen peroxide,
(iii) 8.5 to 9.5 wt.-% ethyl cellulose,
(iv) 15 to 28 wt.-% water,
(v) 0.2 to 0.5 wt.-% dexpanthenol and
(vi) 0.01 to 0.06 wt.-% aroma, in each case relative to the overall mass of the composition.

21. Packaging unit, containing a receptacle with a composition according to claim 1, a dish for receiving the quantity required for a single treatment of the teeth and an applicator for the composition.

22. Agar solution containing
a) 1 to 30 wt.-% gelling agent;
(b) 0.01 to 0.1 wt.-% aromatic compound;
(c) optionally 0.001 to 0.01 wt.-% peroxidase;
(d) buffer solution (ad 100 wt.-%).

23. A method of bleaching teeth comprising the steps of (i) cleaning and drying the teeth to be treated, (ii) applying the composition according to claim 1 to the surface of the teeth; (iii) allowing the composition to dry to form a film; (iv) leaving the film on the teeth for a period of time, (v) removing the film, repeating steps (i) to (v) until the desired bleaching result is achieved.

24. The method of claim 24 wherein the period of time in step (iv) is from 15 to 30 minutes.

25. The method of claim 23, wherein the agar solution contains 5 to 25 wt.-% gelling agent.

26. The method of claim 23, wherein the agar solution contains 10 to 20 wt.-% gelling agent.

27. The method of claim 23, wherein the agar solution contains 0.015 to 0.05 wt.-% aromatic compound.

28. The method of claim 23, wherein the agar solution contains 0.02 to 0.03 wt.-% aromatic compound.

29. The method of claim 23, wherein the agar solution contains o-dianisidine.

30. The method of claim 23, wherein the agar solution contains 0.0015 to 0.003 wt.-% peroxidase.

31. The method of claim 23, wherein the agar solution contains NaK-phosphate buffer.
